(19) United States
(12) Patent Application Publication
(10) Pub. No.: US 2022/0293238 A1
(43) Pub. Date: Sep. 15, 2022

(54) PROPOSAL SYSTEM, PROPOSAL METHOD, AND PROGRAM

(71) Applicant: Aventino, Inc., Yokohama-shi, Kanagawa (JP)

(72) Inventor: Akiko KANEKO, Yokohama-shi, Kanagawa (JP)

(21) Appl. No.: 17/621,690

(22) PCT Filed: Jan. 18, 2021
(86) PCT No.: PCT/JP2021/001405

§ 371 Date: Dec. 22, 2021

(30) Foreign Application Priority Data
Nov. 19, 2019 (JP) 2019-209201

(51) Int. Cl. G16H 20/60; G16H 20/90; G06T 7/00; A61Q 5/00

(52) U.S. Cl. CPC G16H 20/60; G16H 20/90; G06T 7/0016; A61Q 5/00

(57) ABSTRACT

There are provided a proposal system, a proposal method, and a program that evaluate chronological information about a user to make a proposal related to hair care. The proposal system 1 includes a proposal unit 211 that acquires chronological information about the user over a predetermined period of time to generate the proposal based on the chronological information.

### TECHNICAL FIELD

The present invention relates to a proposal system, a proposal method, and a program and relates to, for example, a technique of evaluating chronological information about a user to make a proposal related to hair care.

### BACKGROUND ART

There have been variously proposed methods for proposing products or services to users. For example, there is a known technique for accepting submission of reviews on products or services, scoring the products or services based on the reviews, and presenting the products or services with high ratings to users. In addition, there is a known technique for tracking the action of a user who visits a web site for selling products and displaying on the screen other products assumed to match the preference and interests of the user according to the products browsing history of the user.

Patent Literature 1 discloses a system that acquires user's answers to some questions and proposes supplements suitable for the user based on these answers.

### CITATION LIST

### Patent Literature

Patent Literature 1: JP 6586499 B

### SUMMARY OF INVENTION

### Technical Problem

However, proposing methods based on a third person's subjectivity or information about a user's own preferences and interests are not suitable for proposals that require expert knowledge, such as hair care.

In this regard, the system disclosed in Patent Literature 1 analyzes the answers of a user based on expert knowledge and proposes measures. Specifically, the system identifies the user's current condition and ideal condition based on the answers to questions and proposes measures (supplements) to approach the ideal condition in accordance with a predetermined algorithm. This proposal method is based on the assumption that the user's current condition and ideal condition, and measures are defined in pairs.

However, especially with regard to problems about hair, the factors that affect the transition from the ideal hair condition to an unideal hair condition are diverse and complex, and the influence of each factor changes individually and over time. For this reason, when the goal is to approach the ideal hair condition, pairing the user's current condition and the measures based on the answers to the questions at a certain point in time is not enough to make an appropriate proposal.

The present invention has been made to solve such a problem and to provide a proposal system, a proposal method and a program that are capable of evaluating chronological information about a user to make a proposal related to hair care.

### Solution to Problem

According to an embodiment, a proposal system that makes a proposal related to hair care to a user, the system includes a proposal generation unit that acquires chronological information about the user over a predetermined period of time and generates the proposal based on the chronological information.

According to an embodiment, the proposal system further includes a user-log acquisition unit that acquires an answer to a question about a condition of hair, health, or life of the user a plurality of times over a predetermined period of time, in which the proposal generation unit generates the proposal based on the answer.

According to an embodiment, the proposal system further includes an image analysis unit that acquires an evaluation value of an image of a head of the user a plurality of times over a predetermined period of time, in which the proposal generation unit generates the proposal based on the evaluation value of the image.

According to an embodiment, the proposal system further includes a supplier-log acquisition unit that acquires a record about hair care, medical examination, or medication performed on the user a plurality of times over a predetermined period of time, in which the proposal generation unit generates the proposal based on the record.

According to an embodiment, in the proposal system, the proposal generation unit generates a plurality of the proposals and extracts a part of the plurality of the proposals based on priority assigned to the plurality of the proposals.

According to an embodiment, in the proposal system, the proposal generation unit generates a plurality of the proposals and outputs the plurality of the proposals in accordance with an execution order assigned to the plurality of the proposals.

According to an embodiment, a proposal method of a computer to make a proposal related to hair care to a user, the method includes the steps of acquiring chronological information about the user over a predetermined period of time, and generating the proposal based on the chronological information.

According to an embodiment, a program causes a computer to execute the proposal method.

### Advantageous Effects of Invention

According to the present invention, it is possible to provide a proposal system, a proposal method and a program that are capable of evaluating chronological information about a user to make a proposal related to hair care.

### BRIEF DESCRIPTION OF DRAWINGS

FIG. 1 is a diagram schematically showing a service to be achieved by a proposal system 1;

FIG. 2 is a block diagram showing a hardware configuration of the proposal system 1;

FIG. 3 is a block diagram showing a functional configuration of the proposal system 1;

FIG. 4 is a flowchart showing the operation of the proposal system for sign-up and sign-in for a user;

FIG. 5 is a flowchart showing the operation of the proposal system 1 for acquiring a user log;

FIG. 6 is a flowchart showing the operation of the proposal system 1 for acquiring an image;

FIG. 7 is a flowchart showing the operation of the proposal system 1 for acquiring a supplier log;

FIG. 8 is a flowchart showing the operation of the proposal system 1 for making a proposal to a user;

FIG. 9 is a diagram showing an example of a condition table;

FIG. 10 is a diagram showing an example of a profile input screen;

FIG. 11 is a diagram showing an example of a portal screen;

FIG. 12 is a diagram showing an example of a calendar screen;

FIG. 13 is a diagram showing an example of a question answering screen; and

FIG. 14 is a diagram showing an example of an image acquiring screen.

### DESCRIPTION OF EMBODIMENTS

A proposal system 1 according to an embodiment of the present invention makes a proposal for approaching an ideal hair condition in consideration of the characteristics of various factors that affect hair. The proposal system 1 compares information on how the condition of the hair of a user and the conditions related to health, life, activities and the like have changed from the past to the present (chronological information about the user) with chronological information about the user and about other users having characteristics similar to the user to evaluate and analyze the information. In addition, the proposal system 1 makes a proposal related to hair care for approaching an ideal hair condition based on the evaluation and analysis result. Hereinafter, the proposal system 1 according to an embodiment of the present invention will be described in detail with reference to the drawings.

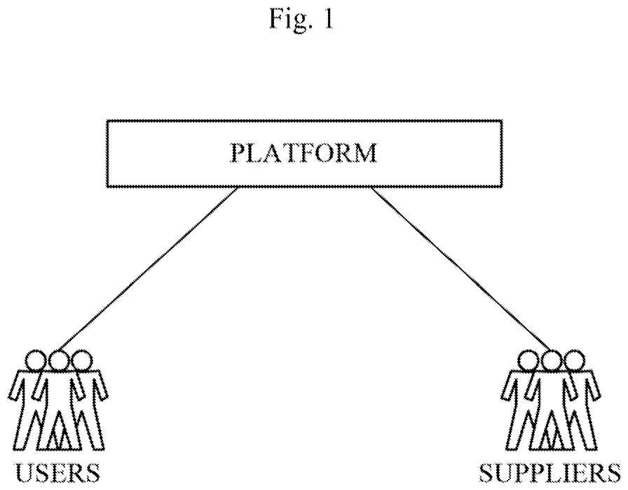

FIG. 1 is a diagram schematically showing a service to be achieved by the proposal system 1. The proposal system 1 provides a platform that meditates between users who care for hair and suppliers that provide the users with products and services assumed to be useful for hair care. The number of the users and the number of the suppliers each can be one or more. The suppliers include cosmetic manufacturers that handle shampoo and other hair care products, pharmaceutical companies that handle hair-related products, health food manufacturers that handle products assumed to be useful for hair care, salons that provide various services assumed to be useful for hair care, medical institutions that provide various hair-related medical services, and the like.

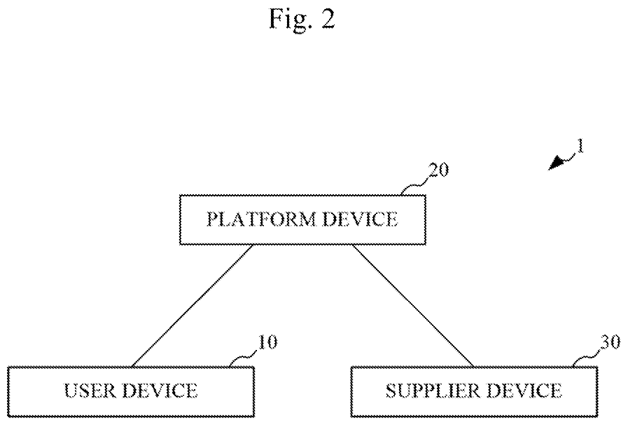

FIG. 2 is a block diagram showing a hardware configuration of the proposal system 1. The proposal system 1 includes a user device 10, a platform device 20, and a supplier device 30. The user device 10 and the supplier device 30 are, for example, information processing devices such as a personal computer (PC), a smartphone, and a tablet. The platform device 20 is, for example, an information processing device such as a server computer or may be an information processing device that is virtually configured using cloud computing or other technologies. Such an information processing device typically includes a central processing device (CPU), a storage device, a display device, an input device, and the like and performs predetermined information processing in accordance with system programs and application programs stored in the storage device. The platform device 20, the user device 10, and the supplier device 30 are communicably connected with each other. This communication can use communication networks such as the Internet, public networks, leased lines, and mobile communication networks.

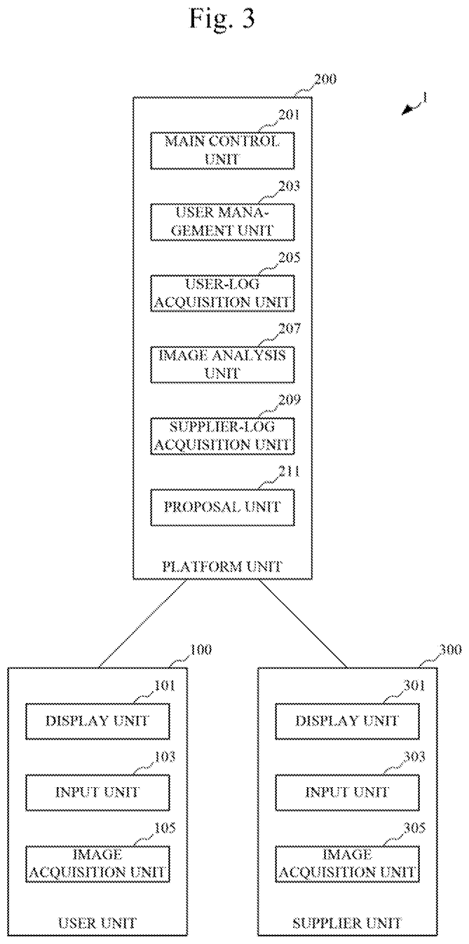

FIG. 3 is a block diagram showing a functional configuration of the proposal system 1. The proposal system 1 includes a user unit 100, a platform unit 200, and a supplier unit 300. Typically, the user unit 100, the platform unit 200, and the supplier unit 300 can be implemented in the user device 10, the platform device 20, and the supplier device 30, respectively, but the present invention is not limited to this embodiment. For example, for some functions of the user unit 100 or the supplier unit 300, the platform device 20 may perform the main processing, and the user device 10 or the supplier device 30 may perform some functions such as input and output.

The user unit 100 includes a display unit 101, an input unit 103, and an image acquisition unit 105.

The display unit 101 controls the screen display of the user device 10. The input unit 103 accepts input from a user using an input device such as a touch panel or a keyboard. The image acquisition unit 105 captures an image of the head of the user with a camera or allows the user to select a captured image file.

The platform unit 200 includes a main control unit 201, a user management unit 203, a user-log acquisition unit 205, an image analysis unit 207, a supplier-log acquisition unit 209, and a proposal unit 211.

The main control unit 201 controls basic operation of a platform application. The user management unit 203 manages a profile and chronological information about the user. The chronological information can contain a user log described later (information to be input by the user), a supplier log (information to be input by a supplier) and images (captured head images that can be acquired either by the user or the supplier). The user-log acquisition unit 205 presents a question to the user and acquires an answer (a user log). The image analysis unit 207 stores and analyzes images acquired by the image acquisition unit 105 and an image analysis unit 305. The supplier-log acquisition unit 209 collects and analyzes a record (a supplier log) about hair care, medical examination, medication and the like performed on the user. The proposal unit 211 generates a proposal for the user based on the chronological information about the user collected by the user-log acquisition unit 205, the image analysis unit 207, and the supplier-log acquisition unit 209.

The supplier unit 300 includes a display unit 301, an input unit 303, and an image acquisition unit 305.

The display unit 301 controls the screen display of the supplier device 30. The input unit 303 accepts input from a supplier using an input device such as a touch panel or keyboard. The image acquisition unit 305 captures an image of the head of a user with a camera in, for example, medical examination or may allow the supplier to select a captured image file.

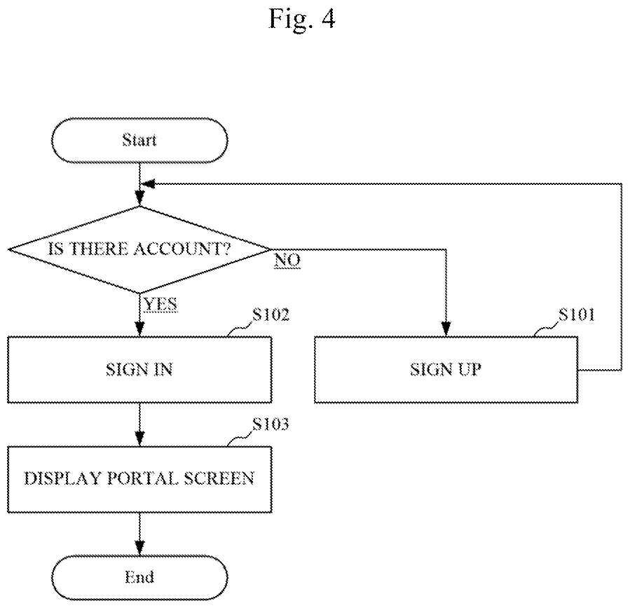

Next, operation of the proposal system 1 according to an embodiment of the present invention will be described with reference to FIGS. 4 to 8.

### <Basic Function>

### (User Management)

FIG. 4 is a flowchart showing the operation of the proposal system 1 for sign-up and sign-in for a user.

### S101: Sign Up

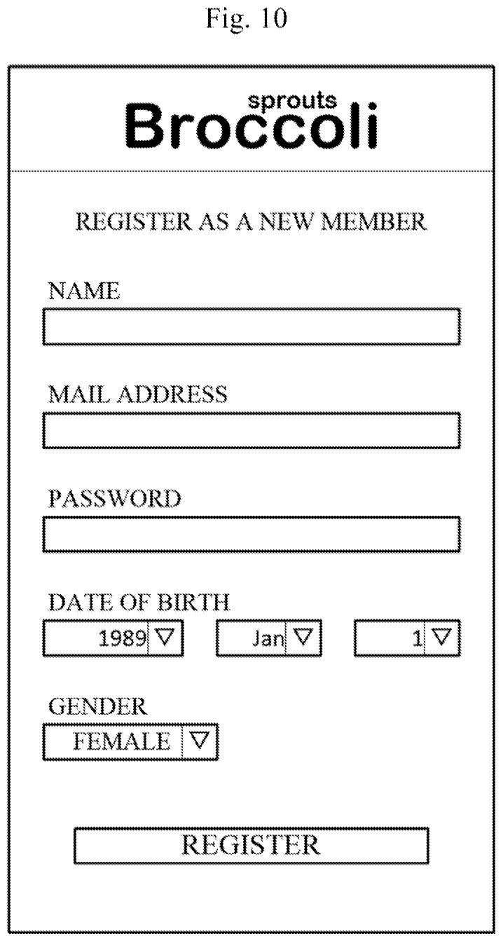

The main control unit 201 requests a user who has not created an account to sign up (to create an account). The user management unit 203 displays a screen for inputting the profile of the user on the display unit 101. FIG. 10 shows an example of a profile input screen. The profile can contain, for example, the user's name, date of birth, gender, mail address, password, and the like. The input unit 103 accepts the profile input by the user and transmits the input content to the user management unit 203.

The user management unit 203 creates a new user account and stores the received profile in association with the account. In addition, the user management unit 203 prepares a storage area for storing chronological information about the user to be collected by the user-log acquisition unit 205, the image analysis unit 207, and the supplier-log acquisition unit 209 in association with this account.

### S102: Sign in

The main control unit 201 requests a user who has an account to sign in (to log in). The user management unit 203 displays a screen for inputting sign-in information (typically, the mail address and the password) on the display unit 101. The input unit 103 accepts the sign-in information input by the user and transmits it to the user management unit 203.

The user management unit 203 allows, when the received sign-in information matches the profile of an existing user, the user to sign in.

### S103: Display Portal Screen

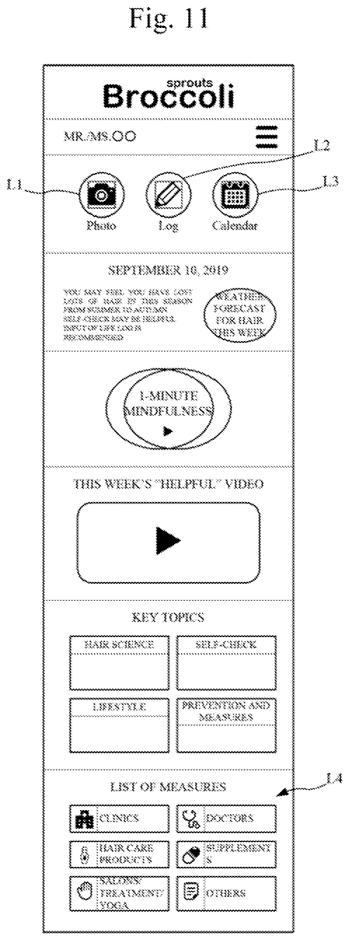

The main control unit 201 displays a portal screen on the display unit 101. FIG. 11 shows an example of the portal screen. In the portal screen, a launcher L1 for displaying an image acquiring screen, a launcher L2 for displaying a question answering screen, and a launcher L3 for displaying a calendar screen are arranged. In addition, a launcher L4 linked to pages for products and services provided by suppliers (for example, sales of hair care products, supplements, and the like, services for consulting or reserving clinics, doctors, salons/treatment/yoga studios, and the like and direct communication described later, and the like can be included) is arranged. Furthermore, daily columns, videos for providing beneficial information to users, links to expert articles provided by information providers including the suppliers, and the like may be arranged.

(Direct Communication with Suppliers)

The platform is capable of providing both the users and the suppliers with a direct communication function for direct communication between a user and a supplier. An implementation example of this function is described.

The main control unit 201 provides the users with a supplier searching screen and a supplier registration screen. The main control unit 201 further provides the suppliers with a user list screen. Furthermore, the main control unit 201 provides both the users and suppliers with a talk screen.

The supplier searching screen is used by a user to search for suppliers that the user is interested in. The main control unit 201 is capable of providing a function of searching profiles of the suppliers with, for example, a category or a keyword. Here, it is assumed that the suppliers have registered their own profiles. The profiles of the suppliers can contain the descriptions about the products and services that they handle and about their fields of expertise. In addition, the main control unit 201 may guide the user to the suppliers' profiles through links from the content (expertise articles or the like) in the site.

The supplier registration screen is used by a user to register suppliers with which the user desires to communicate individually. The user can register favorite suppliers in the user's own list by, for example, pressing a registration button provided in the suppliers' profile pages or the like.

The user list screen is used by a supplier to display a list of users who register the supplier as a favorite. For privacy reasons, it is preferable to control access in such a manner that the supplier can only access the profiles of the users appearing in the user list screen.

The talk screen is used by a user and a supplier to talk (exchange messages) on a one-on-one basis. A user can talk with a supplier registered as a favorite. A supplier can talk with a user appearing in its user list. The talk screen is capable of providing a similar function to a known social networking service (SNS), a short message service (SMS), or the like, but is isolated from an SNS or the like, and has a feature for users to easily talk about a sensitive subject of hair care.

### <Collect Chronological Information>

### (Acquire Users Log)

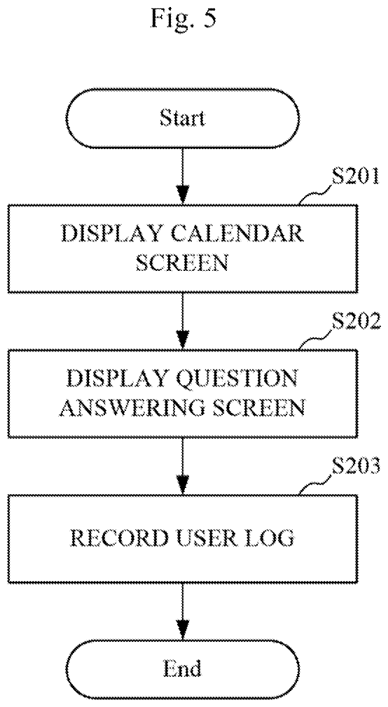

FIG. 5 is a flowchart showing the operation of the proposal system 1 for acquiring a user log.

### S201: Display Calendar Screen

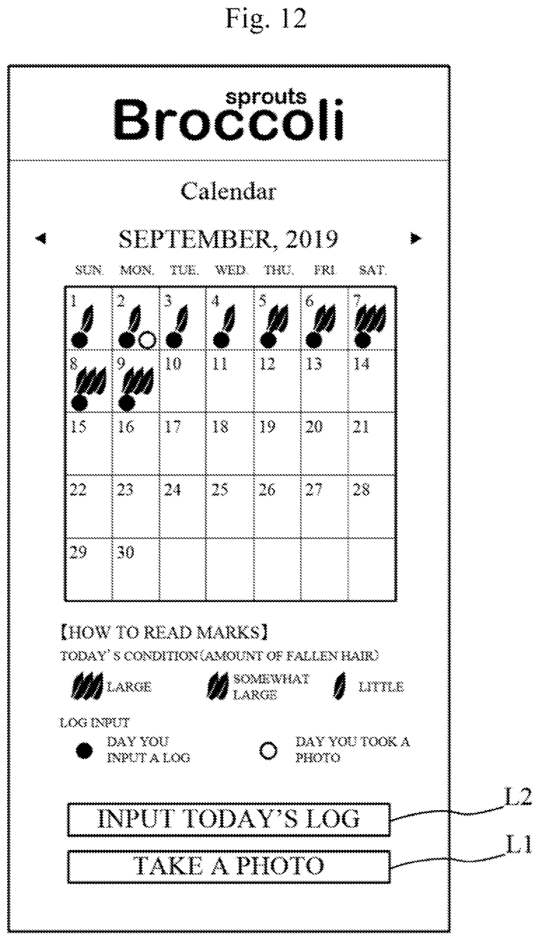

When the launcher L3 is selected, the user-log acquisition unit 205 displays, on the display unit 101, a calendar screen that lists the changes in user logs having been acquired. FIG. 12 shows an example of the calendar screen. The calendar screen shows, on a calendar, “the amount of fallen hair” that a user has answered in the past. In order to improve the perspicuity of the list, “the amount of fallen hair” is divided into three grades in this example to display a leaf icon having a different number of leaves for each grade on the day when the answer has been acquired. Displaying the calendar screen can grasp the changes in the user's hair condition and the like at a glance and have an effect of urging the user to answer the question every day.

In the lower part of the calendar screen, the launcher L2 linked to the question answering screen (S202) for acquiring a user log is arranged. The launcher L1 linked to the image acquiring screen may be arranged.

### S202: Display Question Answering Screen

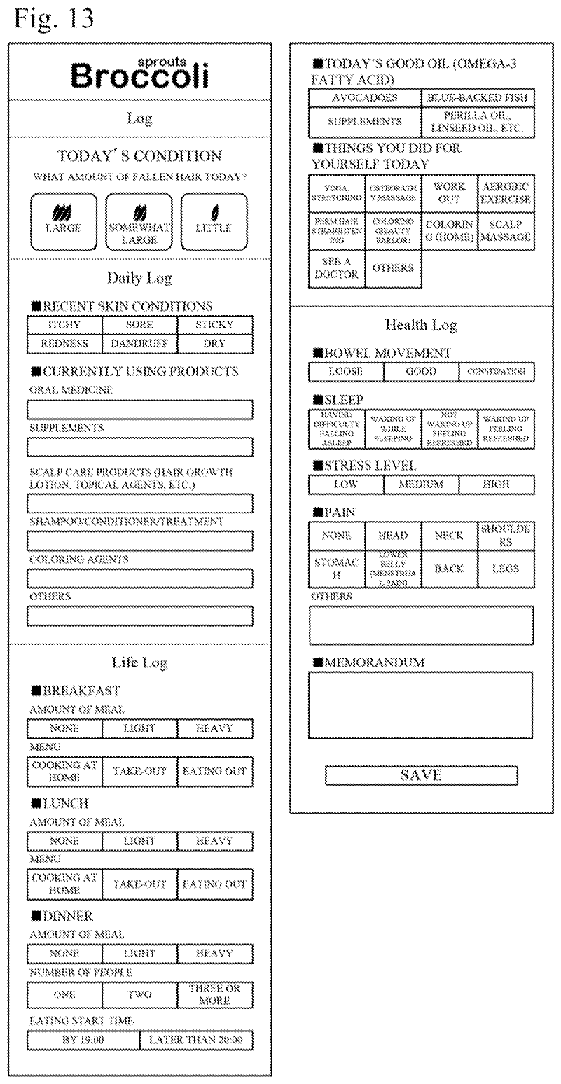

When the launcher L2 is selected, the user-log acquisition unit 205 displays, on the display unit 101, the question answering screen for acquiring a user log. FIG. 13 shows an example of the question answering screen. The user-log acquisition unit 205 acquires, in the question answering screen, a log about hair care performed by the user, daily activities, hair and health conditions, and the like. The user can answer in the question answering screen to, for example, following questions in a selection or free-entry form. The input unit 103 accepts the answers input by the user. The user is allowed to answer at least once a day.
Amount of fallen hair today
Recent skin conditions
Currently using products (oral medicines, supplements, scalp care products, shampoo/conditioner/treatment, coloring agents, others)
Breakfast (amount of food, menu)
Lunch (amount of food, menu)
Dinner (menu, number of people, eating start time)
Oil ingesting status today
Things you did for yourself today
Bowel movement
Sleep
Stress level
Pain
Other things you noticed about your physical condition

### S203: Record User Log

The user-log acquisition unit 205 stores the answers (user log) in association with the user for each day. The user-log acquisition unit 205 preferably encodes the acquired answer to store them. If questions are in selective forms, the answers can be replaced with codes to be stored, such as amount of food: none=0, light=1, heavy=2. If questions are in free-entry forms, words are extracted by, for example, morphological analysis or the like, and the acquired words can be listed and stored. In this manner, by adjusting the user log in a predetermined form, it is possible to easily evaluate the user log, to generate a condition table described later, and perform machine learning.

### (Acquire Image)

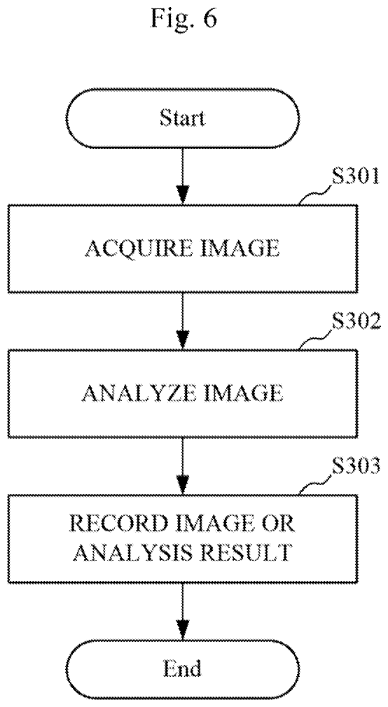

FIG. 6 is a flowchart showing the operation of the proposal system 1 for acquiring an image.

### S301: Acquire Image

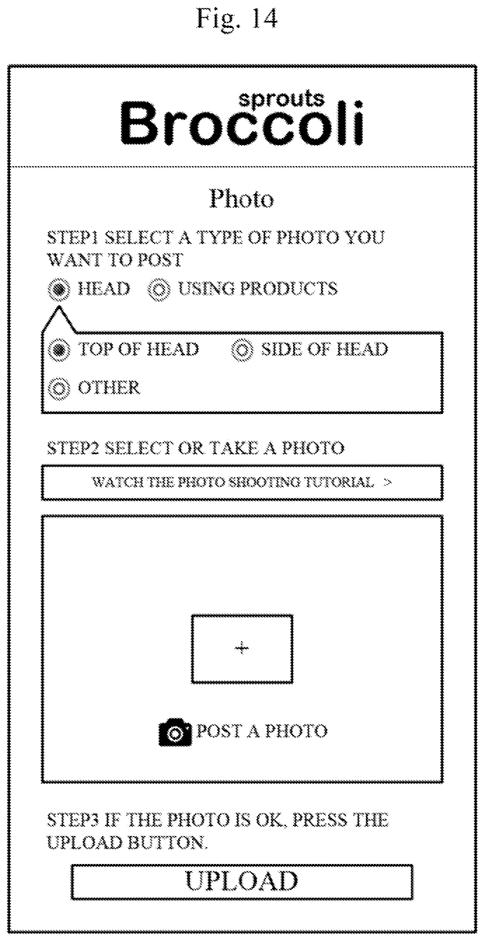

When the launcher L1 is selected, the image analysis unit 207 displays, on the display unit 101, the image acquiring screen for acquiring a captured image of the head of the user or the like. FIG. 14 shows an example of the image acquiring screen. In the image acquiring screen, an interface for the user to select an image captured with the image acquisition unit 105 and to upload it is arranged. Alternatively, an interface for starting the image acquisition unit 105 may be arranged to directly capture and upload an image. When uploading, the image analysis unit 207 may allow the user to specify the type of a captured image. Examples of the type of an image are as follows.
Head (top of head, side of head, others)
Using products

### S302: Analyze Image

The image analysis unit 207 may analyze and evaluate the image acquired in S301. As a method for evaluating a head image, the applicant has disclosed a method in, for example, JP 6582276 B. With this method, it is possible to obtain an evaluation value indicating the change in the hair volume based on an image of the top of the user′ head.

### S303: Record Image or Analysis Result

The image analysis unit 207 stores the image together with the date in association with the user. If the image is analyzed and evaluated, the image analysis unit 207 stores the evaluation value together with the date in association with the user or may store both the image and the evaluation value.

### (Acquire Supplier Log)

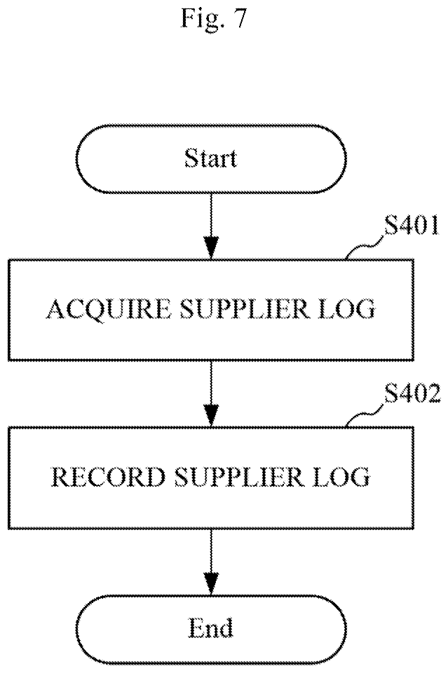

FIG. 7 is a flowchart showing the operation of the proposal system 1 for acquiring a supplier log.

### S401: Acquire Supplier Log

The supplier-log acquisition unit 209 acquires a record (supplier log) about hair care or the like performed by a supplier. The supplier-log acquisition unit 209 displays a log input screen on the display unit 301 of the supplier unit 300. For example, the input unit 303 accepts input of opinions, examination results, medication details, and the like for the user by a doctor who has examined the user and transmits the input to the supplier-log acquisition unit 209. The supplier-log acquisition unit 209 may similarly acquire, from the suppliers, purchase histories of hair care products, supplements, and the like and service providing histories of salons/treatment/yoga studios.

The image analysis unit 207 can display, on the display unit 301, a supplier image acquiring screen for acquiring an image that the supplier has captured the head of the user or the like at the time of medical examination or the like. The supplier image acquiring screen may be based on the image acquiring screen shown in FIG. 14.

### S402: Record Supplier Log

The supplier-log acquisition unit 209 stores the supplier log in association with the user and the date. Similarly to the above user log, the supplier log is preferably adjusted in a predetermined form to be stored.

The image analysis unit 207 may analyze and evaluate the image acquired in S401 and store the image and the evaluation value.

### <Proposal>

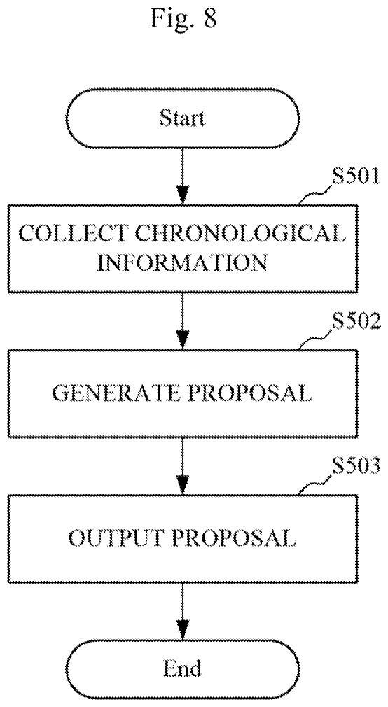

FIG. 8 is a flowchart showing the operation of the proposal system 1 for making a proposal to the user. If, for example, a doctor provides advice on hair care to the user, the advice is generated in accordance with the following procedure.

### S501: Collect Chronological Information

The proposal unit 211 acquires chronological information about the user accumulated in the user management unit 203. The chronological information includes the user logs collected by the user-log acquisition unit 205, the images or the evaluation values collected by the image analysis unit 207, and the supplier logs collected by the supplier-log acquisition unit 209. The proposal unit 211 may specify a period of time for which the information has been acquired to collect the chronological information.

### S502: Generate Proposal

(Method with Condition Table)

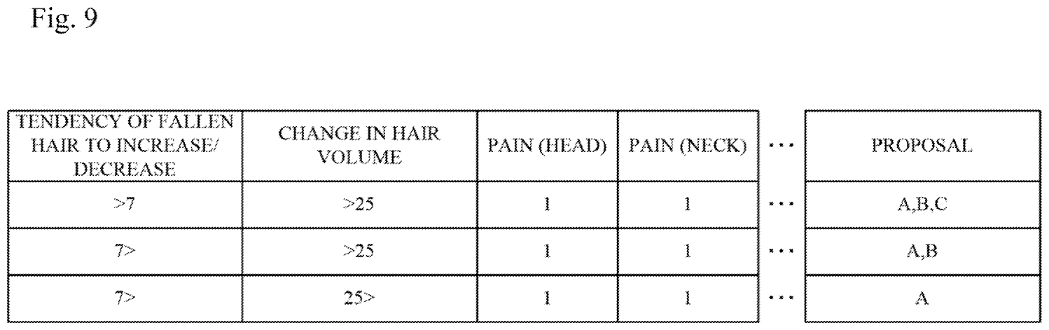

The proposal unit 211 generates a proposal corresponding to the acquired chronological information. For example, the proposal unit 211 can identify an appropriate proposal using a condition table shown in FIG. 9. This condition table is obtained by associating indexes indicating changes in the chronological information with proposals.

The indexes indicating changes in the chronological information can be, for example, as follows.

Tendency of Fallen Hair to Increase/Decrease

For example, “the amount of fallen hair today” in the user log is scored (“large”→2 points, “somewhat large”→1 point, “little”→0 point, and the like), and the increment in the total value of the points over a predetermined period of time is used as an index.

Changes in Hair Volume

For example, the head is divided into six sections, and the hair volume at each section (represented as five grades of Grades 1 to 5) is scored (Grade 5→5 points, Grade 4→4 points, . . . , Grade 1→1 point, and the like) to calculate the sum of the six sections. The increment in the total value of the sums over a predetermined period of time is used as an index.

Body Parts with Health Problems

Pain (1 point) or no pain (0 point) for each body part is used as an index.

The proposal can be, for example, following information.

Products to be used for outside of body (shampoo, topical agents, and the like)

Products to be ingested into body (nutritional supplements, foods, medicines, and the like)

Activities (lifestyle habits and the like)

In the condition table, the correspondence between indexes and proposals may be one-to-one, one-to-many, or many-to-many. That is, if a plurality of indexes is simultaneously observed, a certain proposal may be made. Alternatively, if a certain index is observed, a plurality of proposals may be made.

If a combination of a plurality of proposals is registered, these proposals can be assigned with a priority order or an execution order. For example, if there is a plurality of proposals corresponding to a certain condition and these proposals are assigned with priority, the proposal unit 211 may only extract proposals having higher priority. Specifically, if there are A, B, and C as proposals when a certain index is observed, and when A has been set as “high” priority and B and C have been set as “low” priority, the proposal unit 211 may present A as a proposal. When, for example, a plurality of proposals is assigned with an execution order, the proposal unit 211 may extract proposals in accordance with the execution order. Specifically, if there are A, B, and C as proposals when a certain index is observed, and when A has been set to an execution order “1”, B has been set to an execution order “2”, and C has been set to an execution order “3”, the proposal unit 211 may present proposals in the order of A→B→C.

The condition table can be pre-registered with combinations of indexes and proposals that have been confirmed to be effective based on, for example, past medical examination histories, papers, clinical data, and the like. Thus, it is possible to identify a proposal suitable for the user at present based on the cases of others in which similar characteristics to the user, that is, similar changes in the chronological information have been observed or on the user's own cases in the past.

(Method with Machine Learning)

The proposal unit 211 can generate a proposal using a machine learning technique. That is, the proposal unit 211 learns correlation between changes in chronological information and proposals in accordance with any learning algorithm collectively referred to as machine learning in the learning phase to generate a learned model. Then, the learned model takes the changes in the chronological information as input and outputs a corresponding proposal in the estimation phase.

The learning algorithm used by the proposal unit 211 is not particularly limited. For example, supervised learning can be employed. In this case, in the learning phase, the proposal unit 211 inputs, to a learner, training data constituted by a combination of chronological information about a user (this may be a data set itself constituted by a plurality of pieces of chronological information or indexes calculated from the data set) and a proposal practiced by the user and confirmed to be effective. The learner of the proposal unit 211 repeatedly learns a large amount of training data and gradually recognizes the correlation between the changes in the chronological information and the proposals.

In the estimation phase, the proposal unit 211 inputs the chronological information about a target user to a learned model. The learned model outputs a proposal most suitable for the user as a solution.

As another method, unsupervised learning may be employed. In this case, the proposal unit 211 inputs, to a learner, chronological information about a number of users (this may be a data set itself constituted by a plurality of pieces of chronological information or indexes calculated from the data set) as learning data in the learning phase. The learner of the proposal unit 211 gradually recognizes clusters in a large amount of learning data, that is, a set of similar users.

In the estimation phase, the proposal unit 211 inputs the chronological information about a target user to a learned model. The learned model outputs a cluster to which the user belongs. The proposal unit 211 identifies a representative user belonging to the cluster and outputs a proposal practiced by the representative user and confirmed to be effective as a solution.

### S503: Present Proposal

The proposal unit 211 displays the generated proposal on the display unit 101 of the user unit 100. The proposal may be displayed on, for example, the portal screen (FIG. 11) or on pages providing products and services by the suppliers (pages linked from the launcher L4 in FIG. 11, which are not illustrated), the talk screen, or the like.

Lastly, a specific application example (use case) of the proposal system 1 is described. In this use case, it is assumed that a user is a patient who is aware of fallen hair and a change in hair volume and that a supplier is a doctor who specializes in hair care. The following is a procedure for the doctor to use the proposal system 1 in medical examination.

### (1) First Medical Examination

The doctor conducts a hearing to a patient about the following items during the first medical examination.

Hair and Scalp Conditions

Tendency of fallen hair, changes in total hair volume or exposed skin

Current Status of Hair Care

Whether shampoo, topical agents, coloring agents, perm agents, and the like are used and what kinds of them if used Life style

How to use time for meal, sleep, exercise, and the like and the pace of them

Ingesting Status

Amount of meal, supplements, medicines, and the kinds of them Health problems

Fatigue, stress, gastrointestinal symptoms, skin symptoms, pain, and sleep quality

Anamnesis

Examination Results

Results of blood test from a medical institute and the like

The doctor makes a proposal regarding the following items based on the information acquired at the hearing. This first proposal is decided by the doctor based on data about other patients having characteristics close to the patient.

Products to be used for outside of body (shampoo, topical agents, and the like)

Products to be ingested into body (nutritional supplements, foods, medicines, and the like)

Activities (things the patient can do by themselves, things the patient should request others to do)

### (2) Input Supplier Log and User Log

The doctor inputs, in the log input screen, the opinions obtained as a result of the hearing, the details of the proposal, and the like as a supplier log.

The patient practices the proposal from the doctor and inputs the changes during the practice in the question answering screen and the image acquiring screen every day. The patient inputs information about the following items.

What amount of fallen hair

Health problems

Head image

### (3) Re-Medical Examination

In the next medical examination, the doctor conducts a hearing similarly to (1) and inputs the opinions as a supplier log similarly to (2).

Here, the proposal unit 211 generates a proposal based on the analysis results and the like of the supplier logs, the user logs, and the images input in (1) to (3). The proposal is generated in accordance with a condition table, a learned model, or the like created based on data about others having characteristics similar to the patient or on data about the patient themselves in the past. That is, a proposal practiced by others who's changes in the chronological information are similar to the patient at present or by the patient themselves in the past and confirmed to be effective is output as the proposal for the patient at present. The proposal can be the same as the previous proposal or different. If there is a plurality of proposals, a proposal that is more appropriate (having higher priority) or a proposal that should be practiced earlier (having an earlier execution order) can be output.

Note that, how to define proposals for what change in chronological information in the condition table and the learned model largely depends on intentions and knowledge of the suppliers. For example, according to the knowledge of the inventor, it is important to focus on the following changes in chronological information.

Tendency of days where a patient is aware of a large amount of fallen hair to increase

Tendency of hair volume to decrease or of exposed skin to increase as detected by image analysis

Tendency of health problems to increase

Appearing conditions (season, health problems, change in lifestyle, and the like) that co-occur with tendencies of a large amount of fallen hair, of hair volume in various parts of head to decrease, and of exposed skin to increase

In addition, it is important to define the details of a proposal by focusing on the following facts.

Items in supplier logs or user logs having a difference when poor hair conditions (tendency of days where a patient is aware of a large amount of fallen hair to increase, tendency of hair volume to decrease or of exposed skin to increase as detected by image analysis) and calm hair conditions (with no tendencies described above) are compared to each other

Subsequently, by continuing the processes of (2) and (3), it is possible to continuously make appropriate proposals according to the changes in the chronological information about the patient.

The above use case applied to a specific patient is described as an example.

User: a woman in her 40s

Supplier: a medical institution and a doctor providing various medical services related to hair

### (1) First Medical Examination

A hearing was conducted at the first medical examination as follows.

Hair and Scalp Conditions

Not a large amount of fallen hair. Total hair volume has decreased from her late 30s, and exposed skin has increased.

Current Status of Hair Care

Nothing effective regardless of various trials. Coloring regularly.

Life Style

Having meals regularly. Lack of sleep. Squatting almost every day.

Ingesting Status

Wide variety of foods. Amount is unknown. No supplements and medicines are ingested.

Health Problems

No stress. Having menstruation regularly. Having a backache and migraine. Cold constitution. Sensitive skin. Constipation, nausea, feeling of choked throat.

Anamnesis

Uterine myoma.

Examination results

No problems

The doctor made the proposal based on the hearing result as follows.

Products to be Used for Outside of Body

Topical agent that is not minoxidil

Products to be Ingested into Body

Chinese herbal medicine (for improving digestion)

Multiple vitamin mineral supplements

Amino acid supplements

Activities

No restrictions

### (2) Input Supplier Log and User Log

The doctor input the details of the hearing result and proposal in the first medical examination as a supplier log.

The patient continuously inputs the following user logs and images until the next medical examination.

What amount of fallen hair

Health problems

Head image

### (3) Re-Medical Examination, Input Supplier Log

The above proposal was practiced and the user logs and images were acquired for more than 6 months. As a result, the doctor confirmed that the hair condition of the patient reached a normal range and the condition is maintained for about 6 months. The doctor input the hearing result in the re-medical examination as a supplier log.

Here, a proposal generation unit 305 outputs a proposal for a sole user of a topical agent that is not minoxidil based on the analysis result and the like of the supplier logs, user logs and images input in (2) and (3). Thus, the doctor applied the proposal to the patient. That is, the doctor told the patient to use the topical agent alone.

### (4) Input User Log

The patient continuously input the user logs and images for the same items in (2) until the next medical examination.

### (5) Re-Medical Examination, Input Supplier Log

The above proposal was practiced and the user logs and images were acquired for a year and 7 months. As a result, the doctor observed that the hair volume at the frontal area and around the hair whirl decreased and that the exposed skin around the hair whirl increased. In addition, the doctor heard that the patient has health problems such as being woken up while sleeping, felt stressed, easily swelled, lost exercise habits, and suffered from herpes zoster. The doctor input the hearing result in the re-medical examination as a supplier log.

Here, the proposal unit 211 output the following items as a proposal based on the analysis result and the like of the supplier logs, user logs, and images input in (4) and (5).

Products to be used for outside of body

None

Products to be ingested into body

Amino acid supplements

Probiotics supplements

Activities

Advice to take care of herself physically and mentally, and not to push herself too hard

### (6) Input User Log

The patient continuously input the user logs and images for the same items in (2) for a certain period of time until the next medical examination.

### (7) Re-Medical Examination

The above proposal was practiced and the user logs and images were acquired for a certain period of time. As a result, the doctor confirmed that the hair volume at all areas reached a normal range.

According to the present embodiment, it is possible for the proposal system 1 to acquire and evaluate chronological information about a user to output a proposal. Accordingly, it is possible to make an appropriate proposal related to hair care to approach an ideal hair condition, based on how the user's health and life conditions have changed from the past to the present.

### OTHER EMBODIMENTS

Note that, the present invention is not limited to the above embodiment and can be appropriately modified without departing from the scope thereof. For example, all of the user-log acquisition unit 205, the image analysis unit 207, and the supplier-log acquisition unit 209 acquire chronological information about a user in the above embodiment, but the present invention is not limited thereto, and the processing unit of at least one of them may acquire chronological information.

In addition, each processing described in the above embodiment may be appropriately reordered or replaced with a known method as long as the processing result is equivalent.

In addition, the main control unit 201, the user management unit 203, the user-log acquisition unit 205, the image analysis unit 207, the supplier-log acquisition unit 209, and the proposal unit 211 display information on the display unit 101 or the display unit 301 or acquire information from the input unit 103 or the input unit 303 and acquire images from the image acquisition unit 105 or the image acquisition unit 305 to perform predetermined functions in the above embodiment. However, it is not necessarily required for the proposal system 1 to display and acquire information and images. For example, the main control unit 201, the user management unit 203, the user-log acquisition unit 205, the image analysis unit 207, the supplier-log acquisition unit 209, and the proposal unit 211 may use, as input, information or images received from an outside system or output a processing result to an outside system in any form in a part of or all of the functions. Typically, this is implemented as an application programming interface (API). For example, it is conceivable that the image analysis unit 207 provides an API that inputs an image from the outside and outputs an evaluation result of the image.

In the above embodiment, the present invention is described as a hardware configuration, but is not limited thereto. Any processing can be performed by a central processing unit (CPU) executing a computer program. In this case, the computer program can be stored by various types of non-transitory computer-readable media and provided to a computer. Non-transitory computer-readable media include any type of tangible storage media. Examples of non-transitory computer-readable media include magnetic storage media (such as flexible disks, magnetic tapes, and hard disk drives), optical magnetic storage media (such as magneto-optical disks), Compact Disc Read Only Memory (CD-ROM), CD-R, CD-R/W, and semiconductor memories (such as mask ROM, Programmable ROM (PROM), Erasable PROM (EPROM), flash ROM, and Random Access Memory (RAM)). The program may be provided to a computer using any type of transitory computer-readable media.

Examples of transitory computer-readable media include electric signals, optical signals, and electromagnetic waves. Transitory computer-readable media can provide the program to a computer via a wired communication line (such as electric wires and optical fibers) or a wireless communication line.

### REFERENCE SIGNS LIST

1 Proposal system
10 User device
20 Platform device
30 Supplier device
100 User unit
101 Display unit
103 Input unit
105 Image acquisition unit
200 Platform unit
201 Main control unit
203 User management unit
205 User-log acquisition unit
207 Image analysis unit
209 Supplier-log acquisition unit
211 Proposal unit
300 User unit
301 Display unit
303 Input unit
305 Image acquisition unit
L1, L2, L3, L4 Launcher

1. A proposal system configured to make a proposal related to hair care to a user, the system comprising:
a proposal unit configured to acquire chronological information about the user over a predetermined period of time and generate the proposal based on a change in the chronological information.

2. The proposal system according to claim 1, further comprising a user-log acquisition unit configured to acquire, as the chronological information about the user, an answer to a question about a condition of hair, health, or life of the user a over a predetermined period of time, wherein
the proposal unit is configured to generate the proposal based on the answer.

3. The proposal system according to claim 1, further comprising an image analysis unit configured to acquire, as the chronological information about the user, an evaluation value of an image of a head of the user over a predetermined period of time, wherein
the proposal unit is configured to generate the proposal based on the evaluation value of the image.

4. The proposal system according to claim 1, further comprising a supplier-log acquisition unit configured to acquire, as the chronological information about the user, a record input by a person other than the user about hair care, medical examination, or medication performed on the user over a predetermined period of time, wherein
the proposal unit is configured to generate the proposal based on the record.

5. The proposal system according to claim 1, wherein the proposal unit is configured to generate a plurality of the proposals and extract a part of the plurality of the proposals based on priority assigned to the plurality of the proposals.

6. The proposal system according to claim 1, wherein the proposal unit is configured to combine a plurality of the proposals and output the plurality of the proposals by specifying an execution order.

7. A proposal method of a computer to make a proposal related to hair care to a user, the method comprising the steps of:
acquiring chronological information about the user over a predetermined period of time; and
generating the proposal based on a change in the chronological information.

8. A program causing a computer to execute the proposal method according to claim 7.

9. The proposal system according to claim 1, wherein the proposal unit is configured to calculate an index indicating the change in the chronological information and to output the proposal associated with the index.
